SEQUENCE LISTING (4 sequences)

SEQ ID NO: 1 - DNA, 35 bp, Artificial sequence
  primer
ctgacggctctagaggcttaagtgatttattatgg

SEQ ID NO: 2 - DNA, 34 bp, Artificial sequence
  primer
atcagcatgcactgaattcttactggcgattgtc

SEQ ID NO: 3 - DNA, 36 bp, Artificial sequence
  primer
atcgggatccgattaatactgtagaggtcgacatga

SEQ ID NO: 4 - DNA, 36 bp, Artificial sequence
  primer
aatgaagcttcgacgaatgcgtgaaggggctggagc

(19) United States
(12) Patent Application Publication
(10) Pub. No.: US 2006/0141587 A1
(43) Pub. Date: Jun. 29, 2006

(54) Process for the preparation of L-3, 4-dihydroxyphenylalanine by aerobic fermentation of a microorganism

(75) Inventors: Marco Kramer, Julich (DE); Susanne Kremer-Muschen, Linnich-Gereonsweiler (DE); Marcel G Wubbolts, Sittard (NL)

Correspondence Address: MAYER, BROWN, ROWE & MAW LLP, 1909 K STREET, N.W., WASHINGTON, DC, 20006, US

(73) Assignees: DSM IP Assets B.V., Heerlen (NL); DSM Biotech GmbH, Julich (DE)

(21) Appl. No.: 10/523,370

(22) PCT Filed: Jul. 31, 2003
(86) PCT No.: PCT/EP03/08507

§ 371 Date: Feb. 1, 2006

(30) Foreign Application Priority Data
Aug. 6, 2002 (EP) 02102101.9
Mar. 21, 2003 (EP) 03100730.5

(51) Int. Cl. C12P 13/22; C12N 1/21

(52) U.S. Cl. 435/108; 435/252.3

(57) ABSTRACT

The invention relates to a process for the preparation of L-3,4-dihydroxyphenylalanine wherein L-3,4,-dihydroxyphenylalanine is produced in a fermentation medium by aerobic fermentation of a recombinant microorganism having L-tyro-sine-3-hydroxy-mono-oxygenase activity and at least the metabolic pathways: glycolysis, pentose phosphate pathway, aromatic amino acid pathway, or derivative pathways thereof, which process comprises (i) a growth phase and a production phase, wherein L-3,4-dihydroxy-phenylalanine is produced in the fermentation medium, and 10 (ii) a downstream processing phase, and in which process L-3,4-dihydroxy phenylalanine is produced from a carbon source and the pH is in the range of from 1 to 7 during at least part of the production phase and/or downstream processing phase.

The invention relates to a process for the preparation of L-3,4-dihydroxyphenylalanine, wherein L-3,4,-dihydroxyphenylalanine is produced in a fermentation medium by aerobic fermentation of a recombinant microorganism having L-tyrosine-3-hydroxy-mono-oxygenase activity and at least the metabolic pathways: glycolysis, pentose phosphate pathway, aromatic amino acid pathway, or derivative pathways thereof, wherein the process comprises (i) a growth phase and a production phase, wherein L-3,4-dihydroxy-phenylalanine is produced in the fermentation medium, and (ii) a downstream processing phase.

L-3,4-dihydroxyphenylalanine is also known as L-dopa and is used amongst others in pharmaceuticals for treatment of Parkinson's disease.

Such a process for the production of L-dopa by aerobic fermentation of a microorganism is disclosed by Lee and Xun (1998) Biotechn. Lett., Vol 20, p 479-482 (which process is also described in U.S. Pat. No. 5,837,504). In said process, L-dopa is produced from the precursor L-tyrosine by incubation of cells of a recombinant E. coli DH1 (pAJ221) strain constitutively expressing the hpaBC genes encoding a 4-hydroxyphenylacetate 3-hydroxylase and an FADH₂-NAD oxidoreductase with L-tyrosine under gentle shaking. A drawback of this process is that in the aqueous solution used in these fermentations, L-DOPA is not very stable and is readily oxidized to form black or brown polymerization products. According to the article cited, at page 480, right column at the end of the first full paragraph, such oxidation reaction can only be avoided by the presence of glycerol.

It is the object of the present invention to provide a process for the fermentative production of stable L-dopa.

This object is achieved by the invention by providing a process for the preparation of L-3,4-dihydroxyphenylalanine (as described in the preamble of claim 1) wherein L-3,4-dihydroxy-phenylalanine is produced from a carbon source and wherein during at least part of the production phase and/or downstream processing phase the pH is in the range of from 1 to 7.

It has surprisingly been found that with the process of the invention stable L-dopa can be produced from a carbon source in a commercially attractive way.

The process of the invention is an attractive process for the production of L-dopa, both for practical and for economical reasons, because for example:
1. Stable L-dopa is produced from glucose as a carbon source in the absence of glycerol.
2. A cheap and readily available carbon source, e.g. glucose can be used instead of the cost intensive L-tyrosine, which was used in the process of Lee et al. (1998).

It is to be noticed, that it has been shown in a Japanese patent application (publication number 49-100290; Sep. 21, 1974) that L-dopa may be produced from a glucose source by fermentation of wild-type Pseudomonas cells, the teaching of said reference is clearly directed to increasing the L-dopa production by adding a reducing agent during fermentation in a rich medium (Luria-Bertani medium) and by simultaneous addition of quinic acid or shikimic acid. The problem of stabilizing L-dopa towards (auto)oxidation is not addressed. In the process according to the present invention no addition of a reducing agent is required.

Preferably, in the downstream processing phase of the process according to the invention the L-3,4-dihydroxy-phenylalanine produced is extracted from the fermentation medium and reextracted into a reextraction mixture.

In one embodiment of the invention, the pH of the fermentation medium is kept between 1-7, preferably between 4-7, more preferably between 5-7, in particular between 5.5-6.8, most in particular between 6-6.5 during at least part of the fermentation. The fermentation typically comprises a growth phase and a production phase. The ‘growth phase’ of a fermentation is the phase in which the biomass concentration of the microorganism containing fermentation medium increases. The biomass concentration can be determined by measurement of the optical density of the fermentation broth, i.e. the fermentation medium including the cells of the microorganism, at 620 nm (OD₆₂₀). The ‘production phase’ of a fermentation is the phase in which the product, in this case L-dopa, is produced. The growth and production phase can occur one after the other, but in practice the growth and production phase overlap. Preferably, in this embodiment of the invention, the pH of the fermentation medium is kept between 1-7, preferably between 4-7, more preferably 5-7, in particular between 5.5-6.8, most in particular between 6-6.5 during preferably at least 50% (in time), more preferably at least 65%, even more preferably 80%, in particular 90% of the production phase and most in particular during the entire production phase of the fermentation.

In another embodiment of the invention, the pH of the reextraction mixture used in the downstream processing phase is kept between 1-7, preferably between 4-7, more preferably 5-7, in particular between 5.5-6.8, most in particular between 6-6.5 during at least part of the downstream processing phase, preferably during at least 50% (in time), more preferably at least 65%, even more preferably at least 80%, in particular during at least 90% of the downstream processing phase and most in particular during the entire downstream processing phase.

It is most preferred that the pH of the fermentation medium comprising L-3,4-dihydroxy-phenylalanine and/or the pH of the reextraction mixture comprising L-3,4-dihydroxy-phenylalanine is in the range of from 1 to 7 during the entire production phase of the fermentation and/or during the entire downstream processing phase.

Extraction of L-dopa from the fermentation medium can be performed using standard purification techniques, for example by separation of L-dopa from the fermentation medium via ion exchange resins, chromatography processes, adsorption, filtration, evaporation, reverse osmosis, electrodialysis, etc. A very good way of recovering L-dopa from the fermentation medium is by using adsorption resins to which L-dopa can bind and by subsequently eluting the bound L-dopa from the resins with a suitable reextraction mixture, for example with methanol/HCl (pH=2). Examples of resins, which are capable of binding L-dopa are resins with a hydrophobic interactive surface, for example the adsorption resins XAD-4, XAD-7, XAD-16, XAD-1180 and XAD-2010. These XAD-resins, are commercially available from for example Sigma. Preferably, XAD-16 and XAD-1080 are used for the binding of L-dopa.

Extraction of L-dopa from the fermentation medium can be performed after the fermentation has been stopped, but preferably, L-dopa is extracted during the production phase of the fermentation by so-called in situ product recovery techniques. The use of in situ product recovery techniques for the extraction of L-dopa has the advantage that it is possible to prevent a potential product inhibition in the fermentation. In in situ product recovery, the fermentation medium containing the product (in case of the invention L-dopa) and the cells of the microorganism is pumped over one or more separating devices during at least part of the production phase of the fermentation, preferably during the entire production phase of the fermentation, thereby separating the product from the fermentation medium and the cells. The cells and fermentation medium are recycled back for use in the fermentation. Typically in such an in situ product recovery, first the fermentation medium containing the product and the cells of the microorganism is pumped over a filter to separate the cells from the fermentation medium before the product is extracted from the remaining fermentation medium. The cells and fermentation medium are recycled for use in the fermentation.

Accordingly, in this embodiment of the process according to the invention no intermediate purification steps are needed. The advantages thereof are, for instance, that there will be less loss of product and therefore a higher yield; that there will be less unit operations needed and therefore the process is economically more attractive, etc.

Preferably, in the process according to the invention, the fermentation medium containing L-dopa is pumped over a filter to separate the cells from the fermentation medium, after which the medium is pumped over a second filter to separate the proteins from the other dissolved compounds in the fermentation medium and after which L-dopa is extracted from the remaining fermentation medium.

In in situ product recovery L-dopa can for example be extracted by adsorption to different adsorption resins (such as the resins mentioned above) or by extraction into an extraction mixture. Elution of the resins with a suitable reextraction mixture or reextraction of the L-dopa from the extraction mixture with a suitable reextraction mixture, will give purified L-dopa in the reextraction mixture. Preferably, in situ product recovery comprises the steps of pumping the fermentation broth comprising L-dopa and the cells of the microorganism over a filter to separate the cells from the fermentation medium, extracting L-dopa from the fermentation medium by reactive extraction to an extraction mixture, transferring L-dopa into a reextraction mixture by reextraction, and recycling of the cells and remaining fermentation medium to the fermentation. A particularly suitable form of downstream processing is reactive extraction and reextraction described in WO 00/66253, which is hereby incorporated by reference. In this document the extraction and reextraction of organic substances containing at least one positively charged and/or chargeable nitrogenous group from an aqueous mixture is effected by making use of an extraction agent which contains at least organic compounds of 12 to 18 C-atoms and at least one cation exchanger and by making use of a membrane that is wettable by either the aqueous mixture or by the extraction agent and by reextraction of the organic substances from the extraction agent into an aqueous phase. Preferably, in the process according to the invention, L-dopa is extracted from the fermentation medium by using the reactive extraction method as described in Maass et al. (2002) Bioprocess. Biosyst. Eng., p85-96. Said reactive extraction consists of an organic kerosene phase with the cation selective carrier D₂EHPA (di-2-ethylhexyl-phosphonic acid). The organic phase is separated from the fermentation medium containing L-dopa by a membrane through which L-dopa can be extracted into the organic phase. The organic phase containing L-dopa is subsequently contacted via a membrane with an aqueous stripping phase, including sulphuric acid, and L-dopa is reextracted into the aqueous stripping phase. Preferably, in the process according to the invention, the aqueous stripping phase (i.e. the second mixture) has a pH between 1-7, preferably between 4-7, more preferably 5-7, in particular between 5.5-6.8, most in particular between 6-6.5.

A specifically suitable reactive extraction for the extraction of L-dopa from the fermentation medium is extraction and/or reextraction of L-dopa with so called liquid-liquid centrifuges, optionally combined with other reactive extraction techniques.

In yet another embodiment of the invention the pH is kept between 1-7, preferably between 4-7, more preferably 5-7, in particular between 5.5-6.8, most in particular between 6-6.5 during at least part of the fermentation and during at least part of the downstream processing phase. Preferably, the pH is kept between 1-7, preferably between 4-7, more preferably 5-7, in particular between 5.5-6.8, most in particular between 66.5 during at least 50% (in time), more preferably at least 65%, even more preferably at least 80%, in particular 90%, most in particular during 100% of the production phase of the fermentation and during at least 50% (in time), more preferably at least 65%, even more preferably at least 80%, in particular 90%, most in particular during 100% of the downstream processing phase, preferably, the pH of the reextraction mixture is kept between 1-7, preferably between 4-7, more preferably 5-7, in particular between 5.5-6.8, most in particular between 6-6.5.

With “extraction mixture” is meant a solution, which is suitable for the extraction of L-dopa from the fermentation medium.

With ‘reextraction mixture’ is meant a solution, which is suitable for the extraction of L-dopa from the adsorption resins or extraction medium to which L-dopa has been extracted from the fermentation medium.

With ‘glycolysis or derivative pathways thereof’ is meant the ability of the microorganism to convert glucose or another carbon source into phosphoenol pyruvate (PEP). With ‘pentose phosphate pathway or derivative pathway thereof’ is meant the ability of the microorganism to convert glucose or another carbon source into erythrose 4-phosphate (E4P). A general description of the glycolysis and the pentose phosphate pathway can be found in Stryer, Biochemistry fourth edition 1995, W. H. Freeman and Company, New York. With ‘aromatic amino acid pathway or derivative pathway thereof’ is meant the ability of the microorganism to convert PEP and E4P into L-phenylalanine, L-tyrosine and L-tryptophan. Preferably, the aromatic amino acid pathway is engineered such that L-tyrosine is overproduced. Engineering can for example be performed as described in a review on engineering the aromatic amino acid pathway by Bongaerts et al. (2001), Metabolic Engineering 3:289-300.

For example, the overproduction of L-tyrosine can be achieved by one of the following measures: the absence of a gene encoding a chorismate mutase/prephenate dehydratase (e.g. by deletion of the gene in the microorganism), overexpression of the gene encoding chorismate/prephenate dehydrogenase or by inhibition of the pathway to shikimic acid.

Examples of genes encoding a chorismate mutase/prephenate dehydratase are pheA from Escherichia coli, pheA from Erwinia herbicola, pheA from Haemophilus influenza etc. For example in the microorganism, the gene coding for this enzyme can be deleted by knock-out methods known to the person skilled in the art. Knock-out methods for the inactivation of chromosomal genes in Escherichia coli K-12 are for example described by Datsenko et al. (2000), Proc. Natl. Acad. Sci. USA, Vol. 97: p 6640-6645.

An example of a gene encoding chorismate mutase/prephenate dehydrogenase is the tyrA gene from Escherichia coli.

The pathway to shikimate can for example be inhibited in E. coli by disruption of the global regulator tyrR.

In the process according to the invention, with ‘L-tyrosine-3-hydroxy-mono-oxygenase activity’ is meant the ability to catalyze the hydroxylation of L-tyrosine on the 3-position. Examples of enzymes having this ability are: mono-oxygenases or hydroxylases, for example 4-hydroxyphenylacetate 3-hydroxylase. In a preferred embodiment of the invention L-tyrosine-3-hydroxy-mono-oxygenase activity is an enzyme with hydroxylase activity, more preferably an enzyme with 4-hydroxyphenylacetate 3-hydroxylase activity such as described by Xun et al. (2000), Appl. Environ. Microbiol. Vol. 66 (2), p 481-486. 4-hydroxyphenylacetate 3-hydroxylase activity is the ability to oxidize L-tyrosine to L-3,4-dihydroxyphenylalanine with the co-consumption of molecular oxygen (O₂) and reduced flavin adenine dinucleotide (FADH₂) (see also Xun et al., 2000, Appl. and Envir. Microbiology, 66 (2) pp 481-486 and FIG. 1 below)

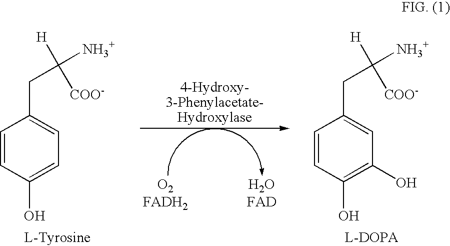

If the microorganism used in the invention does not naturally produce L-tyrosine-3-hydroxy-mono-oxygenase activity, the microorganism can be altered such as to produce this activity, for example by cloning and expression, preferably overexpression, of a gene encoding an enzyme with 4-hydroxyphenylacetate 3-hydroxylase activity (also known as 4-hydroxyphenylacetate 3-hydroxylase) Into a suitable vector into the microorganism. Examples of genes encoding L-tyrosine-3-hydroxy-mono-oxygenases are: PheA encoding phenol hydroxylase from Bacillus thermoleovorans, HpaA encoding 4-hydroxyphenylacetate 3-hydroxylase from Klebsiella pneumonia, hpaB encoding 4-hydroxyphenyalacetate 3-hydroxylase from Escherichia coli. Preferably, the gene encoding a 4-hydroxyphenylacetate 3-hydroxylase is the hpaB gene from Escherichia coli ATCC 11105.

Preferably, in the process according to the invention a microorganism is used that also expresses, and preferably overexpresses, a gene encoding a FADH₂ NAD-oxidoreductase. FADH₂-NAD-oxidoreductase enhances the activity of 4-hydroxyphenylacetate 3-hydroxylase (Xun et al. (2009) Appl. Environ. Microbiol. vol 66: p 481-486). If the microorganism does not naturally have a gene encoding FADH₂-NAD-oxidoreductase or if the expression of the gene is too low, the microorganism can be altered such as to express this gene. For example, if the gene is not naturally present in the microorganism, the gene encoding FADH₂-NAD-oxidoreductase can be cloned in a suitable vector and introduced and subsequently expressed in the microorganism. Genes encoding FADH₂-NAD-oxidoreductase are for example described in Galan et al. (2000), J. Bacteriol. vol 182: p 627-636, for example the hpaC gene from Escherichia coli ATCC 11105B, the fre gene from Escherichia coli, the hpaH gene from K. pneumoni, the hdaB gene from B. pickettii etc.

Preferably, the genes encoding a 4-hydroxyphenylacetate 3-hydroxylase and a FADH₂-NAD-oxidoreductase are overexpressed in the microorganism. Overexpression can be achieved by methods known to the person skilled in the art, e.g. by introducing one or more copies of the gene into the microorganism (e.g. on a muticopy vector or directly into the genome) and/or by placing a suitable promoter before the said gene.

Preferably, in the process according to the invention, a microorganism is used, which overexpresses a gene encoding a feed-back resistant 3-desoxy-D-arabino-heptulosonate-7-phosphate (DAHP) synthase. Preferably, such a microorganism is obtained by deletion of the wild type gene encoding a feed-back regulated 3-desoxy-D-arabino-heptulosonate-7-phosphate synthase in the genome of the microorganism and by complementation of the deletion by a gene encoding an feed-back resistant 3-desoxy-D-arabino-heptulosonate-7-phosphate synthase. For instance, the deletion of the aroF wild type gene encoding an L-tyrosine feed-back regulated 3-desoxy-D-arabino-heptulosonate-7-phosphate synthase in E. coli and the subsequent complementation of the deleted gene by the E. coli gene aroF^{FBR} encoding an L-tyrosine feed-back resistant 3-desoxy-D-arabino-heptulosonate-7-phosphate synthase, is described by Jossek et al. (2001), FEMS Microbiol. Left. Vol. 202: p 145-148.

Cloning of a gene with a known sequence into a suitable vector, introduction into the host microorganism and expression of the gene to produce the desired enzyme (e.g. 4-hydroxyphenylacetate 3-hydroxylase) are standard techniques, which are known to the person skilled in the art (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Suitable vectors are the vectors normally used for cloning and expression and are known to the person skilled in the art. Examples of suitable vectors for expression in E. coli are given e.g. in table 1 in Makrides, S.C., Microbiological Reviews, (1996), Vol. 60. No. 3, p512-538. Suitable vectors for expression in Bacillus are for example described in Wang et al. (1992) Biotechn. Vol 22: p 339-347 and suitable vectors for expression in Corynebacterium are for example described in Deb et al. (1999) FEMS Microbiol. Left. Vol 175(1): p 11-20.

For expression of the gene cloned into the vector, a promoter is usually located upstream of the cloning site in the vector containing the gene encoding the desired enzyme. Suitable promoters are for example the phage lambda PL promoter, the E. coli lac, trp and tac promoters, the SV40 early and late promoters and promoters of retroviral LTRs, to name a few.

Also suitable for use in the invention are promoters, which can be switched on and off, for example the lac promoter, the ara BAD promoter, the tac promoter, the T₇ promoter, the trc promoter and the trp promoter.

For overexpression, a strong promoter, for example the E. coli tac promoter can be used.

The choice of the vector can sometimes depend on the microorganism used as a host. If e.g. a vector with the araBAD promoter is being used, an E. coli host strain that is unable to break down the arabinose inducer (ara⁻), is strongly preferred.

With ‘carbon source’ is meant a compound, which can be converted by the microorganism into E4P and PEP.

Carbon sources which can suitably be used in the process according to the invention are: oligosaccharides and disaccharides, for example maltose, β-galactoside, melibiose, epimelibiose, galactinol, melibitol, galactosylglycerol and trehalose, hexoses, for example D-glucose, D-fructose, D-mannose, L-sorbose and D-galactose, amino sugars, for example N-acetyl-D-glucosamine and D-glucosamine, methylpentoses, for example L-fucose and L-rhamnose, pentoses and trioses, for example L-arabinose, D-arabinose, D-xylose, xylitol, D-lyxose, D-ribose, 2-deoxy-D-ribose and dihydroxyacetone, pentoses in nucleosides and deoxynucleosides, for example cytidine, deoxycytidine, adenosine, deoxyadenosine, uridine, xanthosine, thymidine (deoxyuridine), purine (adenine, hypoxanthine, guanine ribonucleoside), hexuronides, hexuronates and hexonates, for example D-gluconate and D-galactonate, phosphorylated sugars and carboxylates, for example hexose phosphates, and sn-glycerol 3-phosphate, dicarboxylates, for example succinate, fumarate and L-malate, tricarboxylic acids, polyols, for example D-mannitol, D-glucitol, D-sorbitol, galactitol, dulcitol, D-arabitol, ribitol and xylitol, glycerol, two-carbon compounds and fatty acids, for example acetate, fatty acids, glycolate and glyoxylate. Preferably, the used carbon source is glucose.

Examples of microorganism genera, which can suitably be used in the invention are: Escherichia, preferably Escherichia coli, Bacillus, Corynebacterium. In the process of the invention, it is essential that the microorganism has L-tyrosine-3-hydroxy-mono-oxygenase activity; if the microorganism chosen does not naturally have this activity, the microorganism must be altered such that it does have this activity (see above). Preferably, the microorganism used is an Escherichia coli K12 strain, most preferably Escherichia coli W3110 or LJ110. E.g. a very suitable microorganism is Escherichia coli W3110 supplemented with the plasmids pACYCtac aroF^{FBR} tyrA and pJF119EH hpaB hpaC.

The person skilled in the art knows how to conduct a fermentation of a microorganism and knows which fermentation media are suitable for the microorganism. For example suitable fermentation media for fermentation of Escherichia coli are described in: Tanaka et al. (1967), J. Bact. 93:642-648, Pan et al. (1987), Biotechn. Lett. 9:89-94, Gerigk et al. (2002), Bioprocess and Biosystems Engineering 25:43-52.

The invention is illustrated by way of the following examples. However, these examples are not meant to restrict the invention.

### EXAMPLES

Experimental Part

General Procedures

Standard molecular cloning techniques such as DNA isolation, gel electrophoresis, enzymatic restriction modification of nucleic acids, Escherichia coli transformation etc. were performed as described by Sambrook et al., 1989, “Molecular Cloning: a laboratory manual”, Cold spring Harbor Laboratories, Cold Spring Harbor, N.Y. Synthetic oligo deoxynucleotides were obtained from MWG-Biotech AG, Ebersberg. DNA sequence analyses were performed using the chain termination method with dye-labeled dideoxy-terminators.

Plasmid PJF119EH aroF^{fbr} tyrA and pJF119EH hpaBC

As expression vector for the artificial operon encoding a feedback resistant (DAHP) synthase (aroF^{fbr}) and chorismate mutase/prephenate dehydrogenase (tyrA) and the vector consisting an operon 3-hydroxyphenylacetate-4-hydroxylase (hpaB) and a flavin NADH oxidoreductase (hpaC) plasmid pJF119EH was chosen. This vector, constructed by Fürste et al. (1986, Gene, 48: 119-131), is suitable for protein expression in a variety of gram negative bacteria. The pJF119EH system uses the Isopropyl-beta-D-thiogalactopyranoside (IPTG) inducible tac promotor and the lac repressor system (lacI^{Q} gene), which allows to keep the expression of the cloned foreign gene in the absence of the inducer extremely low. Plasmid pJF119EH carries the origin colE1, which is compatible to the origin p15a of plasmid pACYCtac. Gene aroF^{fbr} encoding a feedback resistant DAHP synthase originates from plasmid pJF aroF^{fbr} depicted in Jossek et al., 2001, FEMS Microbiol Lett 202:145-148. A feedback resistant DAHP synthase (AroF (N8K)) is achieved by substituting the amino acid asparagine at position eight of the L-tyrosine feedback sensitive DAHP synthase (AroF) by isoleucine (Jossek et al., 2001, FEMS Microbiol Lett 202:145-148). Gene tyrA originates from the wild type Escherichia coli strain W3110 (ATCC 27325) and the operon consisting of hpaB and hpaC originates from Escherichia coli ATCC 11105 (Davis et al., 1951, Science 114: 459). The construction of the plasmids pJF119EH aroF^{fbr} tyrA and pJF119EH hpaB hpaC are described in examples 1 and 3.

Plasmid pACYCtac aroF^{fbr} tyrA

As expression vector for the genes encoding a feedback resistant (DAHP) synthase (aroF^{fbr}) and a chorismate mutase/prephenate dehydrogenase (tyrA) plasmid pACYCtac was used. Plasmid pACYCtac is based on the vector pACYCtac 184 (Chang and Cohen, 1978, J. Bacteriol. 134; 1141-1156) which is suitable for protein expression in a variety of gram negative bacteria. The pACYCtac184 uses the Isopropyl-beta-D-thiogalactopyranoside (IPTG) inducible tac promotor and the lac repressor system (lacI^{Q} gene), which allows to keep the expression of the cloned foreign gene in the absence of the inducer extremely low. Plasmid pACYCtac184 carries the origin p15a, which is compatible to the origin of colE1 of pJF119EH.

Construction of pACYCtac

For construction of pACYCtac, pACYCtac184 was digested with HindIII plus NruI according to the instructions of the manufacturer (Invitrogen). By gel electrophoresis the 3300 base pair fragment was separated from the approximate 940 base pairs and the 3300 base pair fragment was purified from the agarose gel according to the instructions of the manufacturer (Qiagen). Again with HindIII plus NruI plasmid pZY507 (Weisser et al., 1995, J. Bacteriol. 177; 3351-3345) was opened (according to the instructions of the manufacturer (Invitrogen)) and the smaller fragment of approximately 1600 base pairs was separated by gel electrophoresis, eluted from the gel (according to the instructions of the manufacturer (Qiagen)) and ligated with T4 ligase according to the instructions of the manufacturer (Roche) with the approximately 3300 base pairs of the pACYCtac184 backbone resulting in plasmid pACYCtac.

The construction of plasmid pACYCtac aroF^{fbr} tyrA is described in example 2.

### Example 1

### Construction of Plasmid pJF119EH aroF^{fbr} tyrA

The tyrA open reading frame (ORF) encoding the Escherichia coli chorismate mutase/prephenate dehydrogenase (encoded by nucleotides 5877-4740 of accession number AE000346) was amplified using 5′-CTGACGGCTCTAGAGGCTTAAGTGATTTATTATGG-3′ (with XbaI restriction site underlined) and 5′-ATCAGCATGCACTGAATTCTTACTGGCGATTGTC-3′ (with SphI recognition and cleavage site underlined) as primers (provided by the supplier MWG), and chromosomal DNA of the wild type Escherichia coli strain W3110 as a template. The genomic DNA was isolated according to the manual of a commercial supplier (Macherery and Nagel).

PCR was performed with the Platinum Pfx DNA Polymerase (provided by Life Technologies) according to the instructions of the manufacturer. The resulting DNA amplification product, of approximately 1200 base pairs, was purified by gel extraction (Qiagen) and restricted with XbaI plus SphI according to the manual of the supplier (Invitrogen) and then ligated with T4 ligase according to the instructions of the manufacturer into the vector pJF119EH aroF^{fbr} (Jossek et al., 2001, FEMS Microbiol Lett 202:145-148) according to the instructions of the supplier (Roche)); the vector had already been digested with XbaI plus SphI the same way. Transformation was effected into the strain DH5α (provided by Life Technologies), with selection on LB Broth Base agar (1.5%) plates with ampicilline (100 mg/l) according to the instructions of the manufacturer (Life technologies). Successful cloning was detected by determining the correct sequence of the cloned tyrA gene. The plasmid revealing the correct sequence was designated pJF119EH aroF^{fbr} tyrA.

### Example 2

### Construction of Plasmid pACYCtac aroF^{fbr tyrA}

Plasmid pJF119EH aroF^{fbr} tyrA was opened with MluI plus SphI and the approximate 3000 base pair fragment was isolated by gel electrophoresis and then purified. Plasmid pACYCtac was treated with MluI plus SphI according to the instructions of the manufacturer (Invitrogen), resulting in a 4100 base pair fragment, which was ligated (according to the instructions of the supplier (Roche)) with the 3000 base pair of pJF119EH aroF^{fbr} tyrA treated with MluI plus SphI. Successful cloning was detected by determining the correct insert size. The plasmid revealing the correct insert size was called pACYCtac aroF^{fbr} tyrA. E. coli strain DH5α/pACYCtac aroF^{fbr} tyrA, E. coli DH5α containing plasmid pACYCtac aroF^{fbr} tyrA was deposited under the Budapest Treaty on 23 Jul. 2002 with the DSMZ (Deutsche Sammlung von Mikroorganismen und Zellkulturen, Braunschweig, Germany) under number DSM15110.

### Example 3

### Construction of Plasmid pJF119EH hpaB hpaC

Escherichia coli strain ATCC 11105 was obtained from the American Type Culture Collection (Manassas, Va., U.S.A.). For cultivation of Escherichia coli ATCC 11105 the following medium components were added in 1 l distilled water: 2.0 g yeast extract, 2.0 g Casein hydrolysate, 7.0 g K₂HPO₄, 3.0 g KH₂PO₄, 0.5 g Sodium Citrate 3H₂O, 0.1 g MgSO₄.7H₂O, 1.0 g (NH₄)₂SO₄, 2.0 g Glucose (filter-sterilized). The final pH was adjusted to 7.0. The genomic DNA of Escherichia coli ATCC 11105 was isolated according to the instructions of a commercial genomic purification kit (Macherery and Nagel).

The hpaB and hpaC open reading frames (ORFS) encoding the Escherichia coli ATCC 11105 3-hydroxyphenylacetate-4-hydroxylase and a flavin NADH oxidoreductase (encoded by nucleotides 1112-2674 (hpaB) or 2692-3204 (hpaC) of accession Z29081) was amplified using 5′-ATCGGGATCCGATTAATACTGTAGAGGTCGACATGA-3′ (with BamHI restriction site underlined) and 5′-AATGAAGCTTCGACGMTGCGTGMGGGGC TGGAGC-3′ (with HindIII recognition and cleavage site underlined) as primers, and chromosomal DNA of the ATCC 11105 as a template. The resulting DNA amplification product, of approx. 2100 base pairs, was purified by gel electrophoresis, eluted from the gel according to the instructions of the manufacturer (Qiagen) and restricted with BamHI plus HindIII according to the instructions of the manufacturer (Invitrogen) and then ligated with T4 ligase according to the instructions of the manufacturer (Roche) into the vector pJF119EH (Fürste et al. (1986, Gene, 48: 119-131)), which had already been treated the same way. Transformation was effected into the CaCl₂ treated compentent strain DH5α (provided by Life Technologies), with selection on LB Broth Base agar (1.5%) plates with ampicilline (100 mg/l) according to the instructions of the manufacturer (Life technologies). Successful cloning was detected by determining the gene sequence. PCR was performed with the DNA Polymerase according to the instructions of the manufacturer (Roche).

E. coli strain DH5α/pJF119EH hpaB hpaC, E. coli DH5α containing plasmid pJF119EH hpaB hpaC was deposited under the Budapest Treaty on 23 Jul. 2002 with the DSMZ under number DSM 15109.

### Example 4

### Construction of L-Dopa Producing Strain

As a host for L-dopa production Escherichia coli K12 designated W3110 was chosen (ATCC 27325). Introduction and expression of a copy of genes aroF^{fbr} and tyrA provided on plasmid pACYCtac aroF^{fbr} tyrA (example 2) into the W3110 strain leads to the production of the precursor of L-dopa, L-tyrosine.

Transformation of pACYCtac aroF^{fbr} tyrA (example 2) was performed into the CaCl₂ treated competent strain W3110, with selection on LB Broth Base agar (1.5%) plates with chloramphenicol (25 mg/l) according to the instructions of the manufacturer (Life technologies). The constructed strain was designated W3110 pACYCtac aroF^{fbr} tyrA.

To enable hydroxylation of L-dopa from L-tyrosine plasmid pJF119EH hpaB hpaC was transformed into the CaCl₂ treated competent strain W3110 pACYCtac aroF^{fbr} tyrA and clones were selected on LB Broth Base agar (1.5%) plates with with chloramphenicol (12.5 mg/l) and ampicilline (50 mg/l) according to the instructions of the manufacturer (Life technologies). The constructed was E. coli 3110/pACYCtac aroF^{fbr} tyrA/pJF119EH hpaB hpaC.

### Example 5

### Fermentative Production of L-Dopa from Glucose in Shake Flasks

The mineral medium consisted of Na citrate-3H₂O (1.0 g·l⁻¹), MgSO₄.7H₂O (0.3 g·l⁻¹), KH₂PO₄ (3.0 g·⁻¹), K₂HPO₄ (12.0 g·⁻¹), NaCl (0.1 g·⁻¹), (NH₄)₂SO₄ (5.0 g·⁻¹), CaCl₂.2H₂O (15.0 mg·l⁻¹), FeSO₄.7H₂O (75.0 mg·⁻¹), thiamine.HCl (vitamin B1) (5.0 mg·⁻¹). Additional minerals were added in the form of a trace element solution, (1 ml·l⁻¹), which was composed of Al₂(SO₄)₃.18H₂O (2.0 g·⁻¹), CoCl₂.6H₂O (0.7 g·l⁻¹), CuSO₄.5H₂O (2.5 g·l⁻¹), H₃BO₃ (0.5 g·l⁻¹), MnCl₂.4H₂O (20.0 g·l⁻¹) Na₂MoO₄.2H₂O (3.0 g·⁻¹), NiSO₄.6H₂O (2.0 g·l⁻¹), ZnSO₄.7H₂O (15.0 g·l⁻¹). A stock solution of glucose monohydrate (500 g·⁻¹) was autoclaved separately and added to the sterilized medium to a final concentration of 4 g·l⁻¹ glucose.

From a stock solution chloramphenicol (50 mg/ml), which was solved in ethanol and sterile filtered, a final concentration chloramphenicol of 12.5 mg l⁻¹ was prepared. From a stock solution of ampicilline (50 mg/ml), which was dissolved in water and sterile filtered a final concentration of 50 mg/l was prepared. A single colony of Escherichia coli W3110/pACYCtac aroF^{fbr} tyrA/pJF119EH hpaB hpaC, which was grown over night in mineral medium agar plates (see above for composition), in which 1,5% agar was added, was used to inoculate a 100 ml shake flask containing 10 ml of minimal medium and incubated at 33° C. for 16 hours. About 0.5 ml of this culture was subsequently used to inoculate 50 ml of the same medium in a 500 ml shake flask and incubated at 33° C. and 180 rpm for 24 h. After 3 h, at an OD₆₂₀ nm of approximately 0.2, the cells were induced by adding 0.1 mM Isopropyl-beta-D-thiogalactopyranoside (IPTG). After 24 h culture samples were taken for HPLC analysis of L-dopa concentration.

To perform the HPLC analysis, the HPLC 1100 system of Aegilent (Waldbronn, Germany) with a Diode Array Detector was used. The compounds were measured at a wavelength of 200 nm. A Nucleosil-120-5-C18 column (250×4 mm) from Macherey-Nagel was used as the solid phase. The column was eluted using a gradient starting with eluent A (10 mM H₃PO₄) and eluent B (100% acetonitril). Gradient: 0 min 2% B, 25 min up to 90% B, 27 min to 30 min 2% B. The elution rate was set at 1.2 ml/min, the column temperature was set at 40° C.

To calibrate the HPLC for L-dopa, L-dopa was dissolved in pure water. Under these conditions, a retention time of 4.4 min was observed for L-dopa. HPLC analysis revealed the production of 30 mg/l L-dopa after 24 hours.

### Example 6

### Fermentative Production of L-Dopa from Glucose by Fed-Batch Fermentation

The mineral fermentation medium consisted of Na citrate-3H₂O (1.5 g·⁻¹), MgSO₄.7H₂O (0.9 g·⁻¹), KH₂PO₄ (3.0 g g·⁻¹), NaCl (1 g·⁻¹), (NH₄)₂SO₄ (5.0 g·⁻¹), CaCl₂-2H₂O (15 mg·⁻¹), FeSO₄.7H₂O (112.5 mg·⁻¹), thiamine.HCl (vitamin B1) (7.5 g·⁻¹). Additional minerals were added in the form of a trace element solution, (1.5 ml·l⁻¹), which was composed of Al₂(SO₄)₃·18H₂O (2.0 g·l⁻¹), CoCl₂.6H₂O (0.7 g·⁻¹), CuSO₄.5H₂O (2.5 g·l⁻¹), H₃BO₃ (0.5 g·l⁻¹), MnCl₂·4H₂O (20.0 g·l⁻¹) Na₂MoO₄.2H₂O (3.0 g·⁻¹), NiSO₄.6H₂O (2.0 g·l⁻¹), ZnSO₄.7H₂O (15.0 g·l⁻¹). A stock solution of glucose monohydrate (500 g·l⁻¹) was autoclaved separately and added to the sterilized medium to a final concentration of 20 g·⁻¹ glucose.

From a stock solution chloramphenicol (50 mg/ml), which was solved in ethanol and sterile filtered, a final concentration chloramphenicol of 12.5 mg·l⁻¹ was prepared. From a stock solution of ampicilline (50 mg·l⁻¹), which was dissolved in water and sterile filtered a final concentration of 50 mg·l⁻¹ was prepared.

A single colony of Escherichia coli W3110/pACYCtac aroF^{fbr} tyrA/pJF119EH hpaB hpaC, which was grown over night on LB Broth Base agar (1.5%) plates with ampicilline (50 mg·⁻¹) and cloramphenicol (12.5 mg·l⁻¹) was used to inoculate a 500 ml shake flask containing 50 ml of LB Broth Base with ampicilline (50 mg·l⁻¹) and cloramphenicol (12.5 mg·l⁻¹) and incubated at 33° C. until an OD₆₂₀ₙₘ of approximately 1.0 was reached. To the cultivation 50 ml of glycerol (85%) was added, the cells were filled in 2 ml cryogenic vials (Nalgene) and then stored at −70° C.

For preparation of the preculture 1000 ml shake flasks containing 100 ml mineral medium (see example 5 for composition), except for a glucose concentration of 10 g·⁻¹, was used. Shake flasks were inoculated with 100 μl of frozen glycerol cell stock of E. coli W3110/pACYC tac aroF^{FBR} tyrA/pJF119EHhpaBhpaC and incubated for 14 h at 33° C. 200 ml of the preculture was used for inoculation of the fermentor.

Cultivations in a fermentor were conducted in a 2.0 L bioreactor (Labfors, Infors; Switzerland) with 10% inoculation; which was cultivated at 33° C. The cultivation started at pH 6.7. The mineral fermentation medium (see composition above) was directly sterilized in the bioreactor together with the calibrated pH- and Oxygen-sensors.

pH was controlled by 17.5% NH₄OH (1:2 diluted) and a 5 N KOH solution. A separate glucose feed (500 g·⁻¹) was started when the OD620ₙₘ was higher than 5. At OD620ₙₘ 10, which was reached after about 14 hours Isopropyl-beta-D-thiogalactopyranoside (IPTG) was added to a final concentration of 0.1 mM. After 7.5 h after induction the pH of the medium was reduced from pH 6.7 in steps of pH 0.2 units within 30 minutes to a final pH 5.8.

Biomass (OD620ₙₘ) and L-dopa concentrations were determined by HPLC analysis (see example 5 for HPLC conditions) as a function of time (see: Table 1). The L-dopa concentration at 43 hours was related to 10.

TABLE 1
Fermentation with pH-shift:

| Time [h] | Biomass (OD 620ₙₘ) | relative L-dopa concentration |
|---|---|---|
| 15.75 | 15.6 | 0.18 |
| 16.75 | 20.3 | 1.3 |
| 17.75 | 22.9 | 1.8 |
| 19 | 25.7 | 2.7 |
| 20.5 | 27.4 | 5.3 |
| 22 | 26.7 | 6.8 |
| 24 | 26.5 | 9.5 |
| 39.25 | 33 | 9.6 |
| 43 | 33 | 10 |

### Example 7

### Comparative Example Fermentation without Reduction of the pH

A fermentation was performed describes as in example 6 with the following differences:

The pH was not reduced

At OD₆₂₀ₙₘ 5 (reached after 7 hours) IPTG was added.

Results of the fermentation are shown in Table 2 below. The L-dopa concentration reached after 32 hours was related to 10.

TABLE 2
Fermentation without pH-shift

| Time [h] | Biomass (OD 620 nm) | relative L-dopa concentration |
|---|---|---|
| 4.00 | 1.88 | 0 |
| 6.75 | 5.34 | 0 |
| 8.00 | 9.02 | 0 |
| 9.50 | 14.20 | 0.76 |
| 10.50 | 17.13 | 1.6 |
| 13.75 | 26.00 | 5.3 |
| 24.50 | 37.30 | 13 |
| 26.00 | 41.32 | 11 |
| 28.25 | 37.60 | 10.5 |
| 30.25 | 38.20 | 10.1 |
| 32.00 | 39.10 | 10 |

As can be concluded from Table 1 and Table 2, L-dopa is stable in the fermentation wherein a pH-shift from pH 6.7 to pH 5.8 is applied during the fermentation, whereas L-dopa is less stable in the fermentation at pH 6.7, wherein no pH-shift is applied.

### Example 8

### Reactive Extraction of L-Dopa

A sample of the fermentation broth in which L-dopa is present, prepared as described in example 6, which had been adjusted to pH 7,5 (sample A), was used. 5 ml kerosene containing 10% D₂EHPA (pH acidic) was mixed vigorously with the same volume of sample A for 30 seconds on a vortex mixer. After separation of the two phases (about 10 minutes) the water phase containing L-dopa, the extraction medium (sample B) was stored at room temperature. L-dopa dissolved in the organic phase was reextracted by mixing 2 ml of the organic phase with 1 ml of 1 M KCl (pH=6.3) by shaking vigorously for 30s. After separation of the two phases (about 10 minutes), the water phase containing L-dopa, the reextraction medium (sample C) was stored at room temperature. The samples A, B and C were stored at room temperature for 600 hours and L-dopa concentrations were determined at several points in time (up to 600 hours). L-dopa concentrations were determined by HPLC analysis as a function of time (see: Table 3).

TABLE 3

| | | relative L-dopa | |
|---|---|---|---|
| | relative L-dopa | concentration of | relative L-dopa |
| | concentration of | water phase of | concentration of re- |
| | untreated | fermentation broth | extracted organic |
| | fermentation broth | extracted by | phase by KCl |
| Time | (sample A, pH = | kerosene/D₂EHPA | (sample C, pH = |
| [h] | 7.5) | (sample B, pH acidic) | 6.3) |
| 24 | 0.7 | 0.8 | 0.3 |
| 96 | 0.7 | 0.7 | 0.3 |
| 168 | 0.6 | 0.7 | 0.3 |
| 336 | 0.5 | 0.8 | 0.4 |
| 408 | 0.4 | 0.7 | 0.4 |
| 504 | 0.4 | 0.8 | 0.4 |
| 600 | 0.3 | 0.7 | 0.4 |

From the results it can be concluded that without kerosene/D₂EHPA extraction the L-dopa concentration of fermentation broth decreases during the storage time due to the instability of L-dopa. After extraction of the fermentation broth with kerosene/D₂EHPA (acidic pH) decrease of concentration of L-dopa was nearly prevented, from which it can be concluded that extraction with kerosene/D₂EHPA stabilizes L-dopa. After re-extraction of L-dopa with KCl (pH=6.3) no decrease of L-dopa concentration was observed, from which it can be concluded that re-extraction also increases the stability of L-dopa.

### Example 9

### Stability of L-Dopa

A sample of the fermentation broth in which L-dopa is present, prepared as described in example 6, which had been adjusted to pH 7.5 (sample 1), pH 6.5 (sample 2), pH 5.5 (sample 3) and pH 4.5 (sample 4) was used. The samples 1, 2, 3 and 4 were stored at room temperature for 600 hours and L-dopa concentrations were determined at several points in time (up to 600 hours). L-dopa concentrations were determined by HPLC analysis as a function of time (see: Table 4) with the sample D, i.e. fermentation broth at pH 4.5, related to 10.

TABLE 4

| | fermentation | fermentation | fermentation | fermentation |
|---|---|---|---|---|
| Time | broth at pH 7.5 | broth at pH 6.5 | broth at pH 5.5 | broth at pH 4.5 |
| [h] | (sample 1) | (sample 2) | (sample 3) | (sample 4) |
| 24 | 6 | 7 | 9 | 10 |
| 96 | 6 | 7 | 9 | 10 |
| 168 | 5 | 7 | 9 | 10 |
| 336 | 4 | 5 | 9 | 10 |
| 408 | 3 | 5 | 9 | 10 |
| 504 | 3 | 4 | 8 | 10 |
| 600 | 2 | 4 | 8 | 10 |

Results presented in table 4 show that lower pH values of the fermentation broth increase the stability of L-dopa.

1. Process for the preparation of a L-3,4-dihydroxyphenylalanine, wherein L-3,4-dihydroxyphenylalanine is produced in a fermentation medium by aerobic fermentation of a recombinant microorganism having L-tyrosine-3-hydroxy-mono-oxtgenase activity and having at least the abilities to convert glucose or another carbon source into phosphoenol pyruvate (PEP) and into erothrose 4-phosphate (E4P), and to convert the PEP and E4P into L-phenylalanine, L-tyrosine and L-tryptophan, wherein the process comprises
(i) a growth phase and a production phase, wherein L-3,4-dihydroxy-phenylalanine is produced in the fermentation medium and
(ii) a downstream processing phase,
characterized in that the L-3,4-dihydroxy-phenylalanine is produced from a carbon source and in that at least part of the production phase and/or downstream processing phase the pH is the range of from 1 to 7.

2. Process according to claim 1, wherein in the downstream processing phase the L-3,4-dihydroxy-phenylalanine produced is extracted from the fermentation medium and reextracted into a reextraction mixture.

3. Process according to claim 1, wherein the pH of the fermentation medium comprising L-3,4-dihydroxy-phenylalanine and/or the pH of the reextraction mixture comprising L-3,4-dihydroxy-phenylalanine is in the range from 1 to 7 during the entire production phase of the fermentation and/or during the entire downstream processing phase.

4. Process according to claim 1, wherein L-3,4-dihydroxy-phenylalanine is recovered from the fermentation medium by adsorption resins with a hydrophobic interactive surface and by subsequent elution of the bound L-3,4-dihydroxy-phenylalanine from the resins with a reextraction mixture.

5. Process according to claim 1, wherein L-3,4-dihydroxy-phenylalanine is extracted from the fermentation medium by in situ product recovery.

6. Process according to claim 5, wherein in situ product recovery comprises the steps of pumping the fermentation medium comprising L-3,4-dihydroxy-phenylalanine and the cells of the microorganism over a filter to separate the cells from the fermentation medium, extracting L-3,4-dihydroxy-phenylalanine from the fermentation medium by reactive extraction and transferring L-3,4-dihydroxy-phenylalanine into the reextraction mixture by reextraction, and recycling of the cells and remaining fermentation medium to the fermentation.

7. Process according to claim 1, wherein the recombinant microorganism expresses, preferably overexpresses, a 4-hydroxyphenylacetate 3-hydroxylase.

8. Process according to claim 1, wherein the recombinant microorganism also expresses, preferably overexpresses a gene encoding a FADH₂-NAD-oxidoreductase.

9. Process according to claim 1, wherein the carbon source is glucose.

10. Process according to claim 1, wherein the microorganism is Escherichia coli W3110/pACYCtac aroF^{FBR} tyrA/pJF119EH hpaBhpaC.
